(19) United States
(12) Patent Application Publication
(10) Pub. No.: US 2010/0198336 A1
(43) Pub. Date: Aug. 5, 2010

(54) MEDICAL DEVICES AND METHODS OF MAKING THE SAME

(75) Inventors: JAN WEBER, Maastricht (NL); Brian Brown, Hanover, MN (US)

Correspondence Address: FISH & RICHARDSON P.C., P.O. BOX 1022, MINNEAPOLIS, MN, 55440-1022, US

(73) Assignee: Boston Scientific Scimed, Inc., Maple Grove, MN (US)

(21) Appl. No.: 12/762,197

(22) Filed: Apr. 16, 2010

Related U.S. Application Data
(63) Continuation of application No. 10/440,063, filed on May 15, 2003.

(51) Int. Cl. A61F 2/82; B23H 9/14

(52) U.S. Cl. 623/1.15; 205/665; 427/2.25

(57) ABSTRACT

Medical devices, such as stents, and methods of the devices are described. In some embodiments, the invention features a method of making a medical device including providing a body having an electrically insulating first member defining an elongated lumen, and an electrically conducting second member on a first surface of the first member, removing a portion of the second member, and forming the body into the medical device, e.g., a stent.

### TECHNICAL FIELD

The invention relates to medical devices, such as, for example, stents and stent-grafts, and methods of making the devices.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents and covered stents, sometimes called “stent-grafts”.

An endoprosthesis can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

When the endoprosthesis is advanced through the body, its progress can be monitored, e.g., tracked, so that the endoprosthesis can be delivered properly to a target site. After the endoprosthesis is delivered to the target site, the endoprosthesis can be monitored to determine whether it has been placed properly and/or is functioning properly.

One method of monitoring a medical device is magnetic resonance imaging (MRI). MRI is a non-invasive technique that uses a magnetic field and radio waves to image the body. In some MRI procedures, the patient is exposed to a magnetic field, which interacts with certain atoms, e.g., hydrogen atoms, in the patient's body. Incident radio waves are then directed at the patient. The incident radio waves interact with atoms in the patient's body, and produce characteristic return radio waves. The return radio waves are detected by a scanner and processed by a computer to generate an image of the body.

### SUMMARY

In one aspect, the invention features a method of making a medical device, such as a stent. In some embodiments, the stent includes one or more electrically conductive layers that are unable to carry an electrical current in a closed loop. As explained below, this lack of electrical continuity can enhance the visibility of material present in the lumen of the stent during MRI. At the same time, the stent can be made relatively strong, e.g., the stent is capable of supporting a body lumen.

In another aspect, the invention features a method of making a medical device, such as a stent, including providing a body having an electrically insulating first member defining an elongated lumen, and an electrically conducting second member on a first surface of the first member, removing a portion of the second member and forming the body into the device, e.g., stent. The medical device can be, for example, a catheter, a marker band, a hypotube, or a guidewire.

Embodiments of aspects of the invention may include one or more of the following features. The method includes removing the portion of the second member to expose a portion of the first member. The portion of the second member is removed by electropolishing. The second member defines a non-centric lumen. The first member includes a polymer, a cement, or a ceramic. A thinnest portion of the second member is removed. The method further includes providing an electrically conducting third member on a second surface of the first member. The third member defines a non-centric lumen. The second member defines a non-centric lumen, and the lumens of the second and third members are spaced relative to each other about a perimeter of the body. The second member defines a non-centric lumen, and the lumens of the second and third members are spaced about 180° relative to each other about a perimeter of the body. The second member defines a lumen having a non-circular cross section. The lumen of the second member has an oval cross section or a polygonal cross section. The second member defines a lumen having a circular cross section.

In another aspect, the invention features a method of making a stent, including providing an electrically insulating first tubular member, providing an electrically conducting second tubular member on a surface of the first tubular member, the second tubular member defining a non-centric lumen, removing a portion of the second tubular member to expose a portion of the first tubular member, and forming the first and second tubular members into the stent.

The method can further include providing an electrically conducting third tubular member on a second surface of the first tubular member, and removing a portion of the third tubular member to expose a portion of the first tubular member.

In another aspect, the invention features a medical device, such as a stent, including a body defining a lumen (e.g., a tubular body) including an electrically insulating first member defining a lumen, and an electrically conducting second member on a first surface of the first member, the second member defining a lumen and having multiple thicknesses. The medical device can be, for example, a catheter, a marker band, a hypotube, or a guidewire.

Embodiments of aspects of the invention may include one or more of the following features. The second member defines a non-centric lumen. The second member defines a circular lumen. The second member defines a non-circular lumen. The first member includes a cement, a polymer, and/or a ceramic. The second member includes a non-ferrous material. The stent further includes an electrically conducting third member on a second surface of the first member, the third member defining a lumen. The lumens of the second and third members are displaced relative to each other about a circumference of the body. The third member has multiple thicknesses. The stent further includes a strut having only a portion of the insulating first member and a portion of the conducting third member. The stent further includes a strut having only a portion of the insulating first member and a portion of the conducting second member.

In another aspect, the invention features a method of making a device, such as a stent, including forming a member having an electrically insulating coating into a first structure defining a lumen, the first structure having edges spaced from each other, contacting the edges together, and forming the first structure into the device, e.g., stent.

Embodiments of aspects of the invention may include one or more of the following features. The edges are contacted together by drawing the first structure. The method further includes providing a second structure on a first surface of the first structure, the second structure defining a lumen and having an electrically insulating coating, the second structure further including edges spaced from each other. The edges of the first and second structures are spaced relative to each other about a perimeter.

In another aspect, the invention features a method of making a device, e.g., stent, including forming an electrically conducting first tubular body, removing a first portion of the first tubular body, depositing an electrically insulating material in the first portion, and forming the first tubular body into the device, e.g., stent.

Embodiments of aspects of the invention may include one or more of the following features. The first portion is a seam portion of the first tubular body. The method further includes forming an electrically insulating layer on the first tubular body. The method further includes drawing the first tubular body. The method further includes providing a second tubular body on a surface of the first tubular body. The first and second tubular bodies include seams spaced relative to each other about a perimeter. The seams are spaced about 180° relative to each other.

Embodiments may have one or more of the following advantages. The methods described below can be used to make other medical devices, such as those that include tubes or other enclosing structures, to enhance visibility of material in the devices. The medical devices can be, for example, catheters, marker bands, or hypotubes.

Other aspects, features and advantages of the invention will be apparent from the description of the preferred embodiments and from the claims.

### DESCRIPTION OF DRAWINGS

FIG. 1 illustrates a method of making a stent.

FIG. 2 is a detailed illustration of a portion of the stent of FIG. 1.

FIG. 3A is a cross-sectional view of a strut, taken along line 3A-3A of FIG. 2; and FIG. 3B is a cross-sectional view of a strut, taken along line 3B-3B of FIG. 2.

FIG. 4 illustrates a portion of a method of making a stent.

FIG. 5 illustrates a method of making a stent.

### DETAILED DESCRIPTION

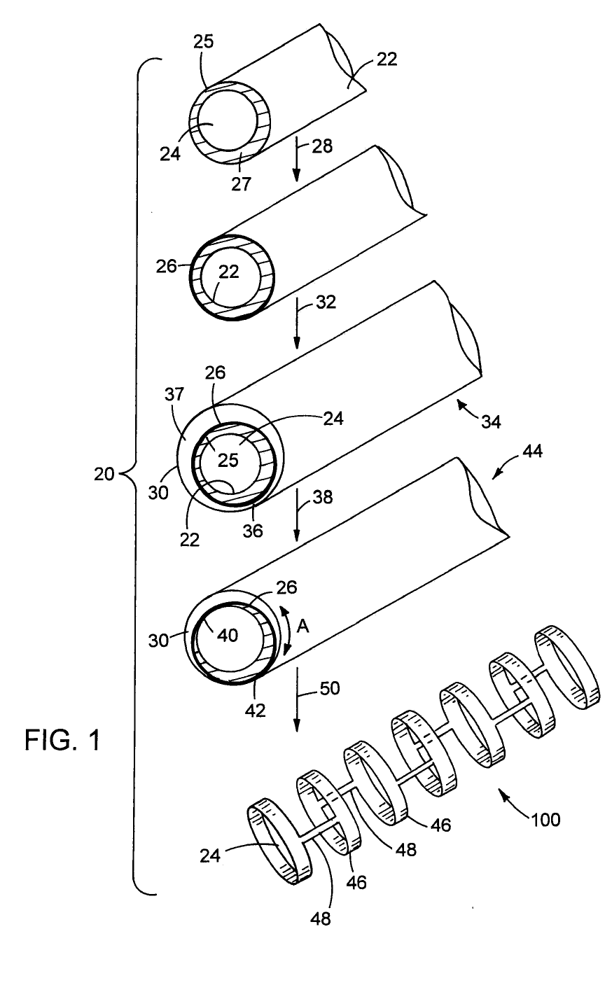

Referring to FIG. 1, a method 20 of making a stent 100 is illustrated. Method 20 is capable of providing a stent that includes electrically conductive portions that are unable to carry an electrical current in a closed loop, e.g., around the circumference of the stent. Consequently, as described more below, the visibility of material, such as blood or a stenosis, present in the lumen of stent 100 during magnetic resonance imaging (MRI) can be enhanced.

Method 20 provides a mechanically strong stent having at least one electrically conductive portion (e.g., layer) interrupted by an electrical insulator. Method 20 includes providing an electrically conductive inner tubular member 22. Inner tubular member 22 has a non-centric lumen 24 such that along a radial cross section, the inner tubular member has a relatively thin portion 25 and a relatively thick portion 27. Next, a layer of electrically insulating material 26 is formed over inner tubular member 22 (step 28), and subsequently, an electrically conductive outer tubular member 30 is formed or placed over layer 26 (step 32) to yield a three-layer tubular member 34. As shown, three-layer tubular member 34 is formed such that inner tubular member 22 and layer 26 are non-centric with respect to outer tubular member 30, e.g., diametrically opposed to lumen 24. As a result, similar to inner tubular member 22, outer tubular member 30 has a relatively thin portion 36 and a relatively thick portion 37.

Next, in step 38, portions of inner tubular member 22 and outer tubular member 30 are removed. As shown, thin portions 25 and 36, are removed to reveal an inner portion 40 and an outer portion 42 of electrically insulative layer 26, respectively. The result is a tubular member 44 having inner tubular member 22 and outer tubular member 30 separated by electrically insulative layer 26, and each member 22 and 30 is interrupted by the electrically insulative layer at portions 40 and 42, respectively. As a result, neither inner tubular member 22 nor outer tubular member 30 can carry an electrical current circumferentially (arrow A) around tubular member 44.

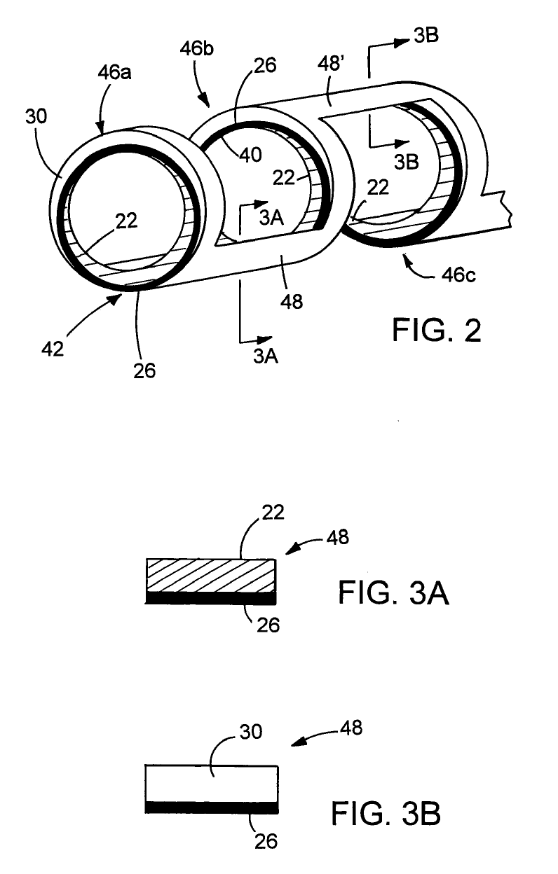

Tubular member 44 is then formed, e.g., by laser cutting, into stent 100 having bands 46 and struts 48 connecting the bands (step 50). In particular, referring to FIGS. 2 and 3, struts 48 are formed at selected locations of bands 46 such that there is no electrical continuity between the bands for an electrical current to flow in a closed loop. As shown, one strut 48 is formed at portion 42 (FIG. 2). Starting at any starting reference point of inner tubular member 22 of band 46a, electrical current can flow to inner tubular member 22 of band 46b via a section of tubular member 22 in strut 48 (FIG. 3A). However, the electrical current cannot flow back to the starting point to close a loop because inner tubular member 22 of band 46b is interrupted by insulative layer 26 at portion 40. Electrical current also cannot flow from outer tubular member 30 of bands 46a or 46b through strut 48 because the strut does not include a portion of the outer tubular member. Similarly, alternatively or in addition to strut 48 shown in FIG. 2, a strut including a portion of insulative layer 26 and a portion of outer tubular member 30 can be formed at portion 40 (as exemplified by strut 48′ between band 46b and 46c). Current cannot flow to form a loop because outer tubular member 30 of bands 46b and 46c are interrupted by insulative layer 26 at portion 42.

Thus, electrical current cannot flow in a loop within a band because conductive tubular members 22 and 30 are interrupted by insulative layer 26. Current also cannot form a closed loop by flowing between bands because struts 48 are formed at selected positions to prevent an electrical current loop from forming.

The lack of electrical continuity within a band and between bands 46 can enhance the MRI visibility of material in the lumen of stent 100. Without wishing to be bound by theory, during MRI, an incident electromagnetic field is applied to a stent. The magnetic environment of the stent can be constant or variable, such as when the stent moves within the magnetic field (e.g., from a beating heart) or when the incident magnetic field is varied. When there is a change in the magnetic environment of the stent, which can act as a coil or a solenoid, an induced electromotive force (emf) is generated, according to Faraday's Law. The induced emf in turn can produce an eddy current that induces a magnetic field that opposes the change in magnetic field. The induced magnetic field can interact with the incident magnetic field to reduce (e.g., distort) the visibility of material in the lumen of the stent. A similar effect can be caused by a radiofrequency pulse applied during MRI.

By forming stent 100 to include electrically conductive portions that cannot form a closed current loop, the occurrence of an eddy current is reduced (e.g., eliminated). Accordingly, the occurrence of an induced magnetic field that can interact with the incident magnetic field is also reduced. As a result, the visibility of material in the lumen of stent 100 during MRI can be enhanced.

Method 20 is described in more detail below.

Referring again to FIG. 1, inner tubular member 22 can be formed of any biocompatible material suitable for MRI, e.g., non-ferromagnetic materials. The biocompatible material can be suitable for use in a self-expandable stent, a balloon-expandable stent, or both. For self-expandable stents, inner tubular member 22 can be formed of a continuous solid mass of a relatively elastic biocompatible material, such as a superelastic or pseudo-elastic metal alloy. Examples of superelastic materials include, for example, a Nitinol (e.g., 55% nickel, 45% titanium), silver-cadmium (Ag—Cd), gold-cadmium (Au—Cd), gold-copper-zinc (Au—Cu—Zn), copper-aluminum-nickel (Cu—Al—Ni), copper-gold-zinc (Cu—Au—Zn), copper-zinc/(Cu—Zn), copper-zinc-aluminum (Cu—Zn—Al), copper-zinc-tin (Cu—Zn—Sn), copper-zinc-xenon (Cu—Zn—Xe), indium-thallium (In—Tl), nickel-titanium-vanadium (Ni—Ti—V), and copper-tin (Cu—Sn). See, e.g., Schetsky, L. McDonald, “Shape Memory Alloys”, Encyclopedia of Chemical Technology (3rd ed.), John Wiley & Sons, 1982, vol. 20. pp. 726-736 for a full discussion of superelastic alloys. Other examples of materials suitable for inner tubular member 22 include one or more precursors of superelastic alloys, i.e., those alloys that have the same chemical constituents as superelastic alloys, but have not been processed to impart the superelastic property under the conditions of use. Such alloys are further described in PCT application US91/02420.

In other embodiments, inner tubular member 22 can include one or more materials that can be used for a balloon-expandable stent. Suitable examples of materials include noble metals, such as platinum, gold, and palladium, refractory metals, such as tantalum, tungsten, molybdenum and rhenium, and alloys thereof. Suitable materials include radiopaque materials, such as metallic elements having atomic numbers greater than 26, e.g., greater than 43, and/or those materials having a density greater than about 9.9 g/cc. In certain embodiments, the radiopaque material is relatively absorptive of X-rays, e.g., having a linear attenuation coefficient of at least 25 cm⁻¹, e.g., at least 50 cm⁻¹, at 100 keV. Some radiopaque materials include tantalum, platinum, iridium, palladium, tungsten, gold, ruthenium, and rhenium. The radiopaque material can include an alloy, such as a binary, a ternary or more complex alloy, containing one or more elements listed above with one or more other elements such as iron, nickel, cobalt, or titanium. Other examples of stent materials include titanium, titanium alloys (e.g., alloys containing noble and/or refractory metals), stainless steels, stainless steels alloyed with noble and/or refractory metals, nickel-based alloys (e.g., those that contained Pt, Au, and/or Ta), iron-based alloys (e.g., those that contained Pt, Au, and/or Ta), and cobalt-based alloys (e.g., those that contained Pt, Au, and/or Ta).

Inner tubular member 22 can include a mixture of two or more materials listed above, in any arrangement or combination.

Inner tubular member 22 including non-concentric lumen 24 can be formed by conventional techniques. For example, inner tubular member 22 can be formed from a solid rod of a selected material, and lumen 24 can be mechanically formed, e.g., by drilling. Alternatively, inner tubular member 22 can be extruded to include a non-concentric lumen. The size of lumen 24 can be determined, for example, by the final thickness desired for inner tubular member 22 after thin portion 25 is removed (step 38).

Next, insulative layer 26 is formed on inner tubular member 22 (step 32). Insulative layer 26 can include any electrically non-conductive and MRI compatible material. Suitable materials include polymers, such as thermoplastics or thermosetting materials. The polymer can enhance the flexibility of stent 100. Examples of polymers include polyolefins, polyesters, polyethers, polyamides and nylons, polyvinyl chlorides, copolymers and terpolymers thereof, or mixtures thereof. Other suitable materials include ceramics, such as titanium oxides, hafnium oxides, iridium oxides, chromium oxides, aluminum oxides (e.g., α-Al₂O₃ or yttria-stabilized alumina), glass ceramic (e.g., Macor™, a blend of fluorophlogopite mica and borosilicate glass from Corning, or Bioglass™ from USBiomaterials), calcium phosphate (e.g., hydroxylapatite), zirconium oxide (e.g., transformation toughened zirconia, fully stabilized zirconia, or partially stabilized zirconia with magnesium or yttrium), feldspathic porcelain, and silicon nitride. Other suitable materials include cements. Examples include glass ionomers (e.g., Glasscor™ or Glassbase™ available from Pulpdent), resin reinforced glass ionomers (e.g., Vitrebond™ from 3M), polycarboxylates (e.g., TylokPlus™ from L. D. Caulk), cyanoacrylates, zinc phosphates, resin composite cements (e.g., filled bisphenol-A-glycidyldimethacrylate resin combined with methacrylics, or RelyX ARC from 3M), and cements used in the field of dentistry. Insulative layer 26 can include a mixture of two or more materials listed above, in any arrangement or combination.

In some embodiments, insulative layer 26 can include an insulating form of the material of inner tubular member 22. For example, inner tubular member 22 can include tantalum or tungsten, and insulative layer 26 can include tantalum oxide or tungsten oxide, respectively. Such embodiments can have relatively low interfacial differences (e.g., stress), which can provide good adhesion between the materials.

The thickness of insulative layer 26 can vary. Generally, insulative layer 26 is sufficiently thick to electrically isolate inner tubular member 22 from outer tubular member 30, and/or to prevent members 22 and 30 from carrying a continuous loop of electrical current. Insulative layer 26 is preferably sufficiently thick to withstand processing tolerances, e.g., handling during manufacturing or removal of portions 25 and 36 without damage. In some embodiments, the thickness of insulative layer 26 can range from about 5 to about 200 nanometers for ceramics or cements, or about 0.1 to about 50 micrometers for polymers.

Insulative layer 26 can be formed on inner tubular member 22 according to a variety of techniques. In some cases, the choice of technique is a function of the materials of insulative layer 26 and/or inner tubular member 22. For example, in embodiments in which insulative layer 26 includes a polymer, an adhesive can be used to bond the polymer to inner tubular member 22. In embodiments in which insulative layer 26 includes an insulating form of a material of inner tubular member 22, techniques, such as plasma ion implantation or heating the inner tubular member in an appropriate (e.g., oxidizing) atmosphere, can be used. Other suitable techniques include thermal spraying techniques, such as plasma arc spraying, chemical vapor deposition, physical vapor deposition, or dipping. In certain embodiments, inner and outer tubular members 22 and 30 can be co-drawn, and insulative layer 26, for example, a polymer, can be formed, e.g., by pouring the liquid or molten polymer into the space defined between the members.

After insulative layer 26 is formed, outer tubular member 30 is formed over the insulative layer to form three-layer tubular member 34 (step 32). In general, materials suitable for inner tubular member 22 are also suitable materials for outer tubular member 30. Outer tubular member 30 can be provided as described above for inner tubular member 22. Stent 100 can include the same or different materials for inner and outer tubular members 22 and 30.

Outer tubular member 30 can be joined to inner tubular member 22 and insulative layer 26 using a variety of methods. For example, similar to inner tubular member 22, outer tubular member 30 can include a non-concentric lumen (not shown) into which inner tubular member 22 and insulative layer 26 are inserted. Members 22 and 30 can be joined together by co-drawing the members. Alternatively or in addition, members 22 and 30 can be joined together using magnetic pulse forming or welding. The use of magnetic forces to deform a work piece is described, for example, in Batygin Yu et al., “The Experimental Investigations of the Magnetic Pulse Method Possibilities for Thin-walled Metal Plates Deformation”, Technical Electro-dynamics, 1990, #5, p. 15-19; and commonly assigned U.S. Ser. No. 10/192,253, filed Jul. 10, 2002. In some embodiments, an adhesive can be applied between insulative layer 26 and outer tubular member 30.

As shown in FIG. 1, tubular member 34 is formed such that lumen 24 of inner tubular member 22 and the lumen defined by outer tubular member 30 are offset (as shown, diametrically offset) relative to the circumference of tubular member 34. Expressed another way, thin portions 25 and 36 are about 180 degrees apart about the circumference of tubular member 34. By offsetting the lumens of inner and outer tubular members 22 and 30, when thin portions 25 and 36 are removed to form tubular member 44 (described below), tubular member 44 can be formed with relatively uniform wall thickness and good structural integrity. In other embodiments, lumen 24 and the lumen defined by outer tubular member 30 (or thin portions 25 and 36) are less than about 180 degrees, e.g., between zero and 180 degrees, apart about the circumference of tubular member 34.

After tubular member 34 is formed, portions of inner and outer tubular members 22 and 30 are removed to prevent the members from carrying an electrical current circumferentially around tubular member 34 (step 38). In certain embodiments, thin portions 25 and 36 are removed such that inner and outer tubular members 22 and 30, respectively, are interrupted by insulative layer 26. Since lumen 24 and the lumen of outer tubular member 30 are offset, the portion of inner tubular member 22 that is removed (e.g., thin portion 25) is compensated by relatively thick portion 37 of the outer tubular member. Similarly, the portion of outer tubular member 30 that is removed (e.g., thin portion 36) is compensated by relatively thick portion 27 of inner tubular member 22. As a result, tubular member 44 has relatively uniform wall thickness and good strength.

Portions of inner and outer tubular members 22 and 30 can be removed by a variety of methods. For example, portions of inner and outer tubular members 22 and 30 can be removed by electropolishing, in which both portions can be removed simultaneously. Since thin portions 25 and 36 are thinner than other portions of members 22 and 30, respectively, techniques, such as electropolishing, that uniformly remove layers of members 22 and 30 will eliminate the thin portions first to expose insulative layer 26. Electropolishing is described, for example, in U.S. Pat. No. 6,375,826. Other suitable methods for removing portions of inner and outer tubular members 22 and 30 include laser cutting, mechanical machining (e.g., drilling), and/or chemical etching combined with a suitable masking technique.

Subsequently, tubular member 44 is formed into stent 100 (step 50). For example, selected portions of tubular member 44 can be removed for the tubular member to define bands 46 and struts 48. The portions can be removed by laser cutting, for example, using an excimer laser and/or an ultrashort pulse laser. Laser cutting is described, for example, in U.S. Pat. Nos. 5,780,807 and 6,517,888. In certain embodiments, during laser cutting, a liquid carrier, such as a solvent or an oil, is flowed through lumen 24. The carrier can prevent dross formed on one portion of tubular member 44 from re-depositing on another portion (possibly providing electrical continuity), and/or reduce formation of recast material on the tubular member. Other methods of removing portions of tubular member 44 include mechanical machining (e.g., micro-machining), electrical discharge machining (EDM), photoetching (e.g., acid photoetching), and/or chemical etching.

In some cases, tubular member 34 can be formed into a stent before portions of inner and outer tubular members 22 and 30 are removed. For example, laser cutting tubular member 34 into a stent can precede electropolishing tubular member 34.

Stent 100 can further be finished, e.g., electropolished to a smooth finish, according to conventional methods. In some embodiments, about 0.0001 inch of material can be removed from the interior and/or exterior surfaces by chemical milling and/or electropolishing. Stent 100 can be annealed at predetermined stages of method 20 to refine the mechanical and physical properties of the stent.

In use, stent 100 can be used, e.g., delivered and expanded, according to conventional methods. Suitable catheter systems are described in, for example, Wang U.S. Pat. No. 5,195,969, and Hamlin U.S. Pat. No. 5,270,086. Suitable stents and stent delivery are also exemplified by the Radius® or Symbiot® systems, available from Boston Scientific Scimed, Maple Grove, Minn.

Generally, stent 100 can be of any desired shape and size (e.g., coronary stents, aortic stents, peripheral vascular stents, gastrointestinal stents, urology stents, and neurology stents). Depending on the application, stent 100 can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Stent 100 can be balloon-expandable, self-expandable, or a combination of both (e.g., U.S. Pat. No. 5,366,504). Stent 100 can be delivered by other actuating mechanisms, such as those that include an electroactive polymer or a pneumatic action.

Stent 100 can also be a part of a stent-graft. In other embodiments, stent 100 can include and/or be attached to a biocompatible, non-porous or semi-porous polymer matrix made of polytetrafluoroethylene (PTFE), expanded PTFE, polyethylene, urethane, or polypropylene. The endoprosthesis can include a releasable therapeutic agent, drug, or a pharmaceutically active compound, such as described in U.S. Pat. Nos. 5,674,242 and 6,517,888; U.S. Ser. No. 09/895,415, filed Jul. 2, 2001; and U.S. Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics.

Still numerous other embodiments are possible.

For example, while described above as tubular, inner member 22, insulative layer 26, and/or outer member 30 can have non-circular cross sections, e.g., non-circular inner and/or outer perimeters. The cross sections can be oval, elliptical, or regularly or irregularly polygonal, having three or more sides. The lumens of inner member 22, insulative layer 26, and/or outer member 30 can be relatively concentric. Furthermore, other arrangements of struts 48 are possible.

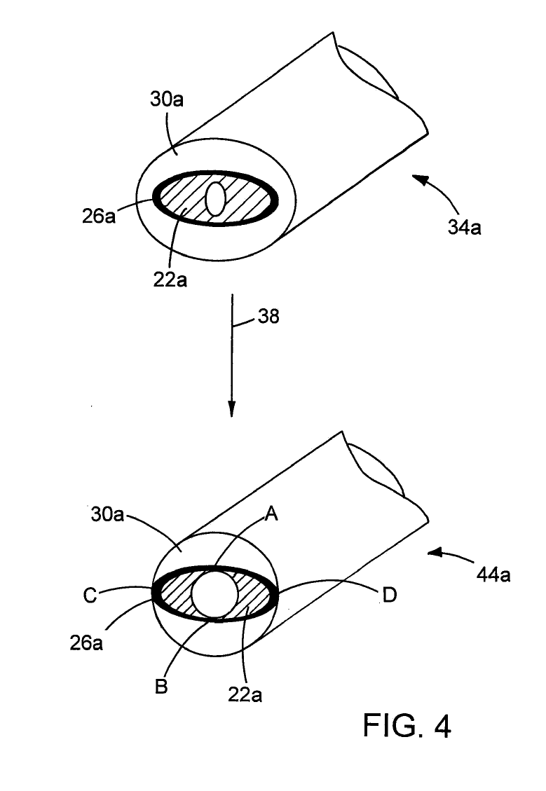

For example, referring to FIG. 4, three-layer member 34a (similar to member 34) includes an inner member 22a, an insulative layer 26a, and an outer member 30a, each having an oval cross section. Inner member 22a, insulative layer 26a, and outer member 30a are generally the same as member 22, layer 26, and member 30, respectively. Three-layer member 34a can be processed as described above (step 38) to remove portions of members 22a and 30a and to prevent members 22a and 30a from carrying a closed loop of electrical current. As a result, a member 44a is formed having member 22a interrupted by insulative layer 26a at two locations (A and B), and member 30a interrupted by the insulative layer at two locations (C and D). Member 44a can be formed into a stent as described above. Struts 48 can be formed in any arrangement at locations A, B, C, and/or D.

While stent 100 is shown including wide, substantially solid bands 46, in other embodiments, bands 46 include a wire shaped in an undulating pattern (as described, e.g., U.S. Pat. No. 6,419,693).

Stent 100 can have fewer or more than the three layers shown in FIG. 1. For example, stent 100 can include insulative layer 26, and inner member 22 or outer member 30.

In some embodiments, stent 100 includes a protective coating on the exterior surface and/or on the interior surface. The coating can be used to enhance the biocompatibility of the stent and/or to protect the stent from corrosion if, for example, the stent includes two different metals. The protective coating can include one or more of the ceramic, polymer, and/or cement described above. More than one protective coatings can be applied.

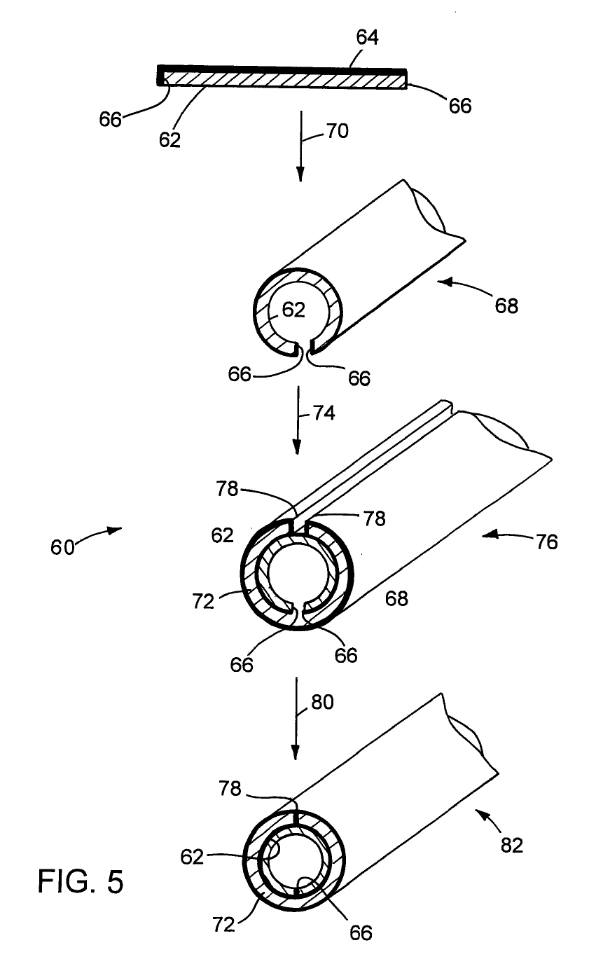

Other methods for making a stent unable to carry electrical current in a closed loop are possible. Referring to FIG. 5, method 60 includes starting with a first sheet 62 of electrically conductive material having an insulative layer 64 on the sheet and on the edges 66 of the sheet. First sheet 62 is then rolled (e.g., around a mandrel) to form a tube 68 having edges 66 spaced apart (step 70). A second sheet 72 (similar to first sheet 62) is formed into a tube and placed over tube 68 to form tubular member 76 (step 74). As shown, the edges 78 of second sheet 72 are spaced apart from each other, and spaced from edges 66, e.g., about 180 degrees. Next, tubular member 76 is reduced in sized (e.g., by drawing) to join edges 66 together, edges 78 together, and sheets 62 and 72 together (step 80). The result is tubular member 82, which can be used to form a stent, as described above (e.g., step 50). Struts 48 can be formed where edges 66 and 78 meet. Sheets 62 and 72 can include the same materials as member 22, and insulative layer 64 can include the same materials as layer 26.

In other embodiments, edges 66 and 78 can be joined together (e.g., by welding) to form tubular member 76 having two seams. After tubular member 76 is reduced in sized (e.g., drawn) to form tubular member 82, the seams can be preferentially removed, e.g., by chemical etching. The removed material can be subsequently replaced with an insulative material. Tubular member 82 can then be formed into a stent as described above.

Method 20 and the embodiments described above can be used to form medical devices other than stents and stent-grafts. For example, method 20 can be used to form filters, such as removable thrombus filters described in Kim et al., U.S. Pat. No. 6,146,404; in intravascular filters such as those described in Daniel et al., U.S. Pat. No. 6,171,327; and in vena cava filters such as those described in Soon et al., U.S. Pat. No. 6,342,062. Method 20 can be used to form guidewires, such as a Meier steerable guidewire, catheters, and hypotubes. Method 20 can be used to form vaso-occlusive devices, e.g., coils, used to treat intravascular aneurysms, as described, e.g., in Bashiri et al., U.S. Pat. No. 6,468,266, and Wallace et al., U.S. Pat. No. 6,280,457. Method 20 can also be used in surgical instruments, such as forceps, needles, clamps, and scalpels.

All publications, applications, references, and patents referred to in this application are herein incorporated by reference in their entirety.

Other embodiments are within the claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A stent, comprising:
a tubular body defining a lumen, the body comprising
an electrically insulating first member, and
an electrically conducting second member on a first surface of the first member, the second member defining a lumen and having multiple thicknesses.

18. The stent of claim 17, wherein the second member defines a non-centric lumen.

19. The stent of claim 17, wherein the second member defines a circular lumen.

20. The stent of claim 17, wherein the second member defines a non-circular lumen.

21. The stent of claim 17, wherein the first member comprises a polymer or a ceramic.

22. The stent of claim 17, wherein the second member comprises a non-ferrous material.

23. The stent of claim 17, further comprising an electrically conducting third member on a second surface of the first member, the third member defining a lumen.

24. The stent of claim 23, wherein the lumens of the second and third members are displaced relative to each other about a circumference of the body.

25. The stent of claim 17, wherein the third member has multiple thicknesses.

26. The stent of claim 23, further comprising a strut consisting of a portion of the insulating first member and a portion of the conducting third member.

27. The stent of claim 17, further comprising a strut consisting of a portion of the insulating first member and a portion of the conducting second member.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A medical device, comprising:
a body defining a lumen, the body comprising
an electrically insulating first member, and
an electrically conducting second member on a first surface of the first member, the second member having multiple thicknesses.

40. A stent, comprising:
a tubular body including in at least a circumferential portion thereof a circumferentially continuous, non-conducting material, and a circumferentially non-continuous, conducting material.

41. The stent of claim 40, wherein the thickness of the non-conducting material is substantially circumferentially constant.

42. The stent of claim 40, comprising first and second non-continuous, conducting material on the inner and outer surfaces of the non-conducting material.

43. The stent of claim 40, wherein the conducting material has variable thickness.

44. The stent of claim 40, further comprising a strut consisting of a portion of the non-conducting material and a portion of the conducting material.

45. The stent of claim 40, wherein the conducting material defines a non-centric lumen.
